(19) United States
(12) Patent Application Publication
(10) Pub. No.: US 2023/0346357 A1
(43) Pub. Date: Nov. 2, 2023

(54) A COMBINED SAMPLE COLLECTION AND FILTRATION DEVICE

(71) Applicant: VIDYA HOLDINGS LTD, London (GB)

(72) Inventors: Marko Dorrestijn, London (GB); David R. Klug, London (GB); Vladimir Turek, London (GB); Stefan Leo Van Workum, London (GB)

(73) Assignee: VIDYA HOLDINGS LTD, London (GB)

(21) Appl. No.: 18/021,981

(22) PCT Filed: Aug. 20, 2021
(86) PCT No.: PCT/GB2021/052171

§ 371 Date: Feb. 17, 2023

(30) Foreign Application Priority Data
Aug. 21, 2020 (GB) 2013087.8

(51) Int. Cl. A61B 10/00; G01N 1/40

(52) U.S. Cl. CPC A61B 10/0051; G01N 1/4077; G01N 2001/4088

(57) ABSTRACT

A combined sample collection and filtration device is provided for collecting and filtering a fluid sample. The device comprises: a sample collection location; a filter module for removing particulate matter from the fluid sample; the filter module having bubble point and a burst pressure; and one or more voids in communication with the sample collection location for accommodating sample during pressurisation such that the pressure within the device remains below the bubble point and the burst pressure of the filter module.

The present invention relates to improvements in or relating to a combined sample collection and filtration device and, in particular, a combined sample collection and filtration device for collecting and filtering a fluid sample.

A fluid sample such as a blood, saliva or urine sample is often collected in a non-sterile environment such as a home, workplace, domestic or retail setting by an unskilled user for diagnostic or research purposes. Collected sample is either analysed immediately or stored and transported for later analysis. The analysis can take a variety of forms, including a sandwich assay. In a sandwich assay, a labelled detection reagent is combined with the sample causing the detection reagent to bind to target species such as a particle, molecule, complex, DNA, protein etc, within the fluid sample. The label-detection reagent-target particle complex then binds to a capture component located in an analysis zone to complete the sandwich assay. The presence of a completed sandwich assay within the analysis zone can be detected through the detection of the label attached to the detection reagent.

Systems for collecting and analysing fluid sample exist in the literature and often involve a disposable cartridge and a reader. Bioassays are performed on the sample collected in certain disposable cartridges which are then inserted into a reader to detect specific biomarkers. In order to identify the presence or absence of a specific biomarker in a fluid sample, a device also needs to interface with its reader and the reader must, in turn, be configured to access the sample and acquire data. Data acquired by the reader is either in the form of raw data such as a measurement of luminescence or it may be a binary result indicating the presence or absence of the predetermined biomarker.

Devices used for the collection of fluid sample at the point-of-care such as disposable cartridges are usually utilised by unskilled operatives therefore such devices need to be intuitive to use and should be resilient. A further requirement is that the fluid sample must be collected in an appropriate volume in order to stop excess sample leaking out of the device or getting in the user's way or contaminating the reader into which the cartridge will be inserted. The device should also be aseptic to prevent contamination of the sample itself.

It may be advantageous that the fluid sample is processed prior to analysis or storage to remove particulate matter. Particles within the sample may interfere with detection methods during analysis or reduce shelf-life of the collected sample.

Methods of filtering fluid samples exist in the literature. However, such methods rely on independent filtering components that form part of complex laboratory workflows. There remains a need in the art for an aseptic device configured to both collect and filter a fluid sample in a non-sterile environment such as a home, workplace, domestic or retail setting by an unskilled user in a manner that does compromise either shelf-life or downstream analysis.

It is against this background that the present invention has arisen.

According to an aspect of the present invention, there is provided the device comprising: a sample collection location; a filter module for removing particulate matter from the fluid sample; the filter module having a bubble point and a burst pressure; and one or more voids in communication with the sample collection location for accommodating sample during pressurisation such that the pressure within the device remains below the bubble point and the burst pressure of the filter module.

According to further aspect of the present invention, there is provided the device comprising: a sample collection location; a filter module for removing particulate matter from the fluid sample; the filter module having a bubble point or a burst pressure; and one or more voids in communication with the sample collection location for accommodating sample during pressurisation such that the pressure within the device remains below the bubble point or the burst pressure of the filter module.

According to another aspect of the present invention there is provided a combined sample collection and filtration device for collecting and filtering a fluid sample, the device comprising: a sample collection location; a filter module for removing particulate matter from the fluid sample; the filter module having a bubble point; and one or more voids in communication with the sample collection location for accommodating sample during pressurisation such that the pressure within the device remains below the bubble point of the filter module.

In the context of the present invention, the term void is used to describe a space or volume within the device that is in communication with the sample collection location so that, when the device is closed, the compressible gas present in the void can be compressed or expelled in order to ensure that the pressure at the filter module does not exceed the bubble point. The void can be completely defined by the structure of the device or it may be partially defined by the fluid sample. The voids may be a single, simple volume or it may consist of a plurality of volumes that may be interlinked or each may be in direct communication with the sample collection location. The communication between the void and the sample may be direct fluid communication in which the edge of the void is defined by the surface of the fluid sample. Alternatively, the void and the sample may be separated by a deformable or movable barrier so that the sample can impact the size or shape of the void, i.e. be in communication with the void, but without a direct fluid communication.

The sample collection location provides a “landing site” for the sample. Related sample collection structures, such as straws, funnels, swabs etc., through which the sample might be collected can interface with the sample collection location, in use. The sample collection location also provides for the metering of the sample so that only the required volume of the sample is pressurised and introduced into the device.

The filter module has a membrane with a bubble point that is related to one or more of the pore size, material from which the filter is made, including any coatings applied to the contact surfaces and the polarity of the filter. If the filter module is made up from a plurality of different filters, provided in a layered configuration, then the bubble point of the filter module as a whole will be dependent on the layer with the highest bubble point from which the filter module is constructed.

The sample may be a saliva sample or a urine sample. Saliva and urine samples differ from blood samples as they are samples of bodily fluid that can be non-invasively collected. If the sample is a saliva sample, the filter module may be further configured to remove mucins from the sample.

The saliva sample deposited in the sample collection location will not be pure liquid. It will include particulate matter; mucins and bubbles entrained within the sample or on the surface of the sample. The filter module removes at least some of the particulate matter entrained in the saliva sample. The filter module may also remove air bubbles and/or mucins entrained in the saliva sample.

The filter module is provided as part of the sample collection and filtration device because it is advantageous to filter the sample as soon as it is collected because the interface between air and liquid provided by air bubbles attracts proteins and may accelerate their degradation or reduce sample shelf life. Other particles such as cells and proteins may reduce shelf life or even interfere with detection.

The provision of one or more voids helps to accommodate the sample during the closure of the device and prevents the pressure from exceeding the bubble point of the filter to substantially reduce the number of bubbles that are introduced into the sample as a result of excessive pressure at the filter. If bubbles are introduced into the sample, they can move through the device with sample and interfere with all aspects of the downstream microfluidics including potentially blocking channels and also degrade the read out by scattering light. This makes the device suitable for use at the point of care by an unskilled operative as the voids mitigate against the use of excessive force to close the device which could otherwise result entering in bubbles within the fluid sample. The filtration of the sample occurs consistently irrespective of user input.

Unregulated pressure within the device can have other downstream detrimental consequences. Fluid may be forced through vents or capillary stops that are intended to stop or reduce flow. By limiting the maximum pressure in the system a narrower range of pressures that the system can encounter allows design of definitive flow stops which would otherwise be impossible to guarantee without pressure limitation. The chance of unwanted leaks is also reduced. The reproducibility of the device is improved.

There may be a plurality of voids and these voids may be separated by a plurality of partitions. These partitioned compressible volumes are small enough that surface tension forces dominate gravitational forces such that the liquid sample does not move off the filter membrane, even if the lid is inverted during lid closure or immediately thereafter as it takes time for the pressure to drive the sample through the membrane. This should work in any orientation including upside down where as a single larger void could leave only gas against the membrane which would stop the filtration process.

The void, or each void where more than one is present, comprises a sealed volume that is compressed upon closure of the device. The provision of the void, or each void in embodiments where more than one void is provided, as a sealed, compressible volume more strictly manages the location of the sample as the void changes size and shape to accommodate the sample, rather than the sample moving into the void. This also reduces the risk of sample remaining in the void rather than moving through the device.

The device may further comprise a compressible absorptive pad. The absorptive pad may further filter/process the liquid sample but also defines the amount of sample required.

The pad may be provided in the sample collection location or it may be accommodated in one or more of the voids. This later configuration is especially appropriate where a single void is provided.

The compressible absorptive pad may be removable. The absorptive pad may be removable and placed in the mouth of the donator or it may be attached to a separate component such as a stick or the lid/plunger.

The sample collection device may further comprise a closure with a one-way latch to ensure the device is an aseptic closed system. Once the sample has been provided and the device is closed, it cannot be opened again. It prevents the device from being used multiple times and ensures that it is a single use item. This prevents contamination of the sample with other samples as multiple samples will never be present within the sample collection location. The one-way latch would retain the closure or lid in the compressed (closed) position once depressed to ensure any excess sample is contained.

The filter module may comprise a plurality of filter-membranes wherein porosity is graded to prevent membrane clogging. The filter module defines the output composition of the fluid sample. The sample could contain particulate matter such as mucins, cells, bacteria, food etc. which will inevitably block the membrane pores at varying rates depending on the sample.

Multiple layers are used in order to help filter out larger particulate matter and avoid clogging. A single membrane with low porosity would be easily clogged by larger particles, such as cells and proteins, filtering out these larger particles prior to passing the sample through lower porosity membranes prevents this issue.

The sample collection device may further comprise a support structure residing between the filter membrane(s) and the compression compartment.

This support structure ensures that the filter module does not tear or is not damaged. Filter membranes can be brittle and may rupture or tear if the pressure is not applied evenly across the membrane as a whole. The support structure may be a series of grooves or ridges that are selected such that they do not interfere with the onward flow of the fluid sample.

The sample collection device may further comprise a guard. The guard can be provided to protect the filter from the user. The guard can be positioned upstream of the filter module, for example, in the sample collection location so that the user does not have direct access to the filter module.

The filter membrane(s) of the filter module can have a positive wetting coefficient and wicks in the liquid sample forming a seal.

The positive wetting coefficient of the filter membrane ensures that, as the liquid sample comes into contact with the filter membrane, it wets the surface and through the filter membrane ensuring that no bubbles become entrained in the sample at the interface with the filter module. The positive wetting coefficient may be inherent to the filter material or it may be provided by a coating or treatment.

The sample collection device may further comprise a check-valve in fluid communication with the filter module. The inclusion of a check valve after the filter module would stop liquid flowing back through the filter.

The sample collection device may further comprise a stabilising agent. The stabilising agent can be provided on the filter membrane(s) or one or more of the walls of the sample collection location. Alternatively, the stabilising agent can be provided either in liquid form downstream of the filter membrane, in the form of a microarray of droplets or mixed with the detection reagent.

This ensures that the stabilisers mix with the sample. This contrasts with commercial devices that require users to shake or invert stabiliser and sample to mix them.

The stabilising agent may pre-wet the filter membrane. This may also ensure the filter membrane remains wet. Mixing with a stabiliser extends the shelf life of the sample making it more amenable to transport and postage or more extreme conditions.

The stabilising agent may be wet or dry. It may be comprised of a cocktail of protease inhibitors that are provided in a solubilised state.

The sample collection device may further comprise a detection reagent. The detection reagent can be provided in the sample collection location, in one or more voids, within the filter module, or downstream of the filter module.

The stabilising agent and detection reagent may be co-located. In this context, the term co-located means that the stabilising agent and the detection reagent are mixed so that they are be applied together to the same location. Consequently, mixing the detection reagent with the stabilising agent means the detection reagent does not need to be printed or manually mixed in at a later point.

The capture component may be an antibody. Alternatively or additionally, the capture component could be a nucleic acid such as DNA, RNA, mRNA or microRNA, or chemically modified nucleic acid; it could be a protein, or a modified protein; or a peptide; or a polymer; it could be a hormone; or a tethered small molecule configured to capture a protein. In some embodiments, the capture component may be a non-specific capture component such as saliva or polylysine. The detection reagent, which can be a secondary antibody, and can be bound with a label can be disposed in various configurations.

The detection reagent can be selected from a peptide, a protein, a protein assembly, an oligonucleotide, a polynucleotide, a modified oligonucleotide, a modified polynucleotide, an aptamer, a morpholino, a small molecule, a cell, a cell membrane, a viral particle, a glycan, a conjugated solid particle, a conjugated solid bead or a cofactor; wherein the detection reagent may be bound with a label.

The label may be, but is not limited to, one or more of the following: a luminophore, a fluorophore, a phosphor, a chemiluminescent molecule, a molecule that exhibits Rayleigh scattering or Raman scattering, an up-conversion particle. an enzyme and its substrate that produces a colorimetric signal; a metallic or inorganic particles e.g. nanoparticles, a polycyclic aromatic hydrocarbon, a metalized complex, a quantum dot or an ion. The ion may be an atomistic ion or a salt of an organic molecule.

The detection reagent may drive an immune reaction for analysis of the sample. The label can be attached to the detection reagent. Additionally or alternatively, the detection reagent may comprise an antibody. In some instances, the detection antibody can be fluorescently labelled.

The one or more voids may further comprise a plunger element for separating fluid sample from gas in the one or more voids and for preventing the fluid sample from flowing off the filter module. This embodiment demonstrates a further means of decoupling the user's action from the pressure by not relying on the user's force but on stored energy within the device which is released upon actuation/closure of the device. This would allow a very controlled force—the lid may comprise small air vents to the ambient.

The one or more voids may further comprise an air vent configured to allow air to escape the device.

The device may further comprise a deformable barrier to the one or more voids for separating fluid sample from gas in the one or more voids and for preventing the fluid sample from flowing off the filter module. The one of the voids may comprise an air vent configured to allow air to escape the device.

One of the voids may further comprise a bladder of compressible gas for separating fluid sample from gas in the one or more voids and for preventing the fluid sample from flowing off the filter module. To keep the liquid against the filter (as it adds parts and/or complexity) a lid with controlled compliance (deformation) can be used instead of compressible gas volume to reduce pressure.

This invention demonstrated herein could be described as positive or driving pressure designs which can generate a larger pressure differential across the filter membrane(s) than alternate designs, such as that disclosed in JPS6245305 that incorporate a negative or suction force as the pressure differential. These alternate designs are limited to a pressure differential of about 1 bar where greater pressure differentials to drive fluid filtration through the membrane can be achieved with this invention.

The present invention will now be further described, by way of example only, with reference to the accompanying drawings in which:

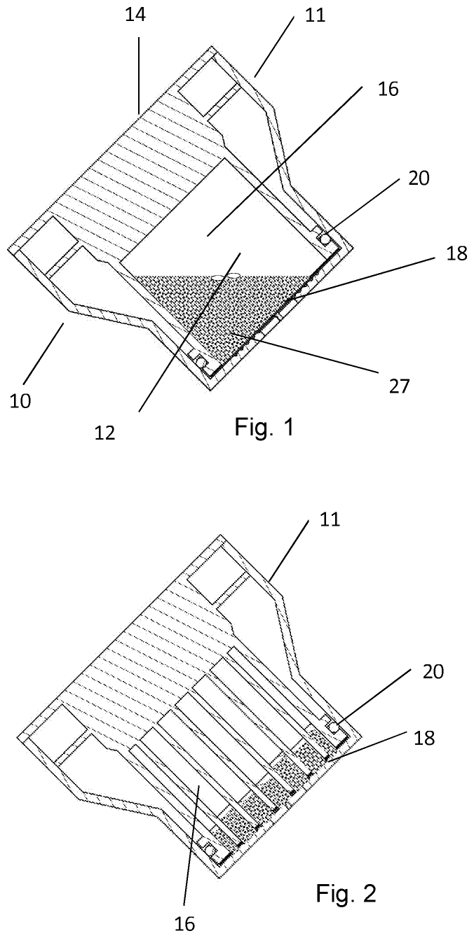

FIG. 1 shows a cross section through a combined sample collection and filtration device with a single void;

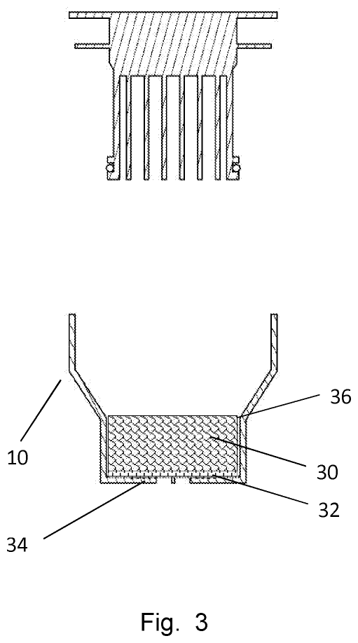

FIG. 2 shows a cross section through a combined sample collection and filtration device with a plurality of voids;

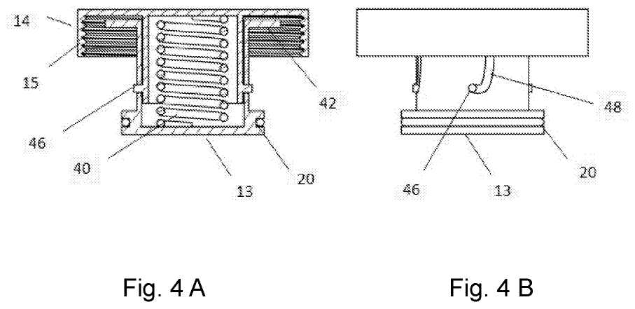

FIG. 3 shows a cross section through a combined sample collection and filtration device with a compressible absorptive material;

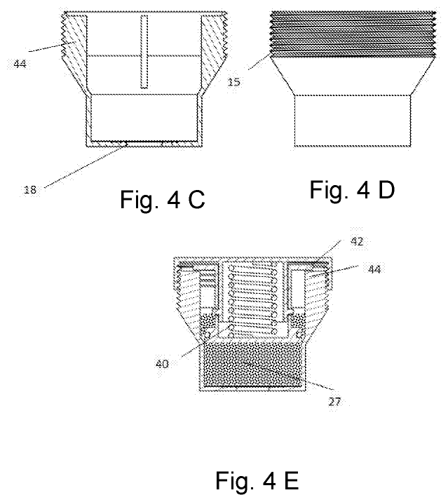

FIGS. 4A to 4E show various cross section and side views of a combined sample collection and filtration device in which the pressure applied to the filter module has been entirely decoupled from the user's actions;

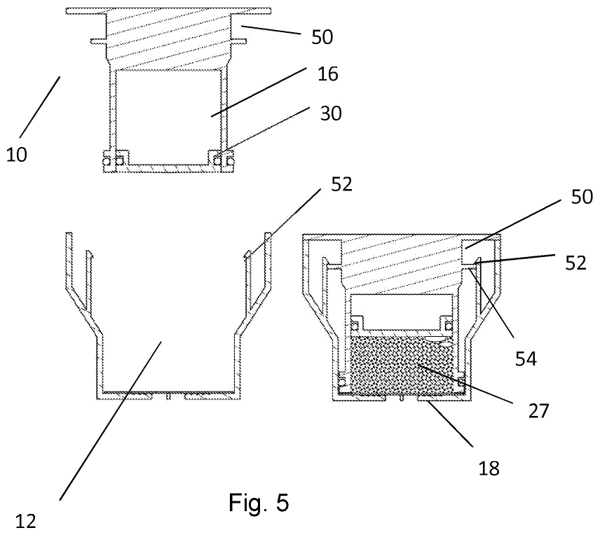

FIG. 5 shows a cross section through a combined sample collection and filtration device with a second plunger;

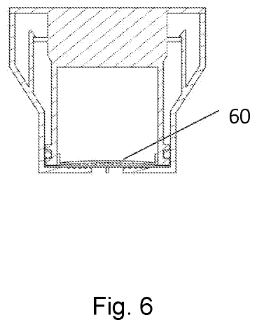

FIG. 6 shows a cross section through a combined sample collection and filtration device with a deformable barrier;

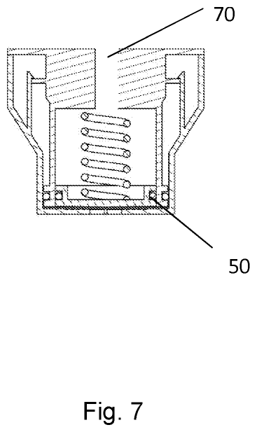

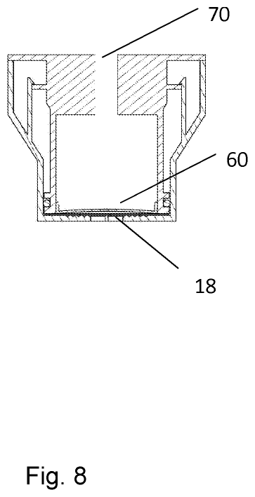

FIGS. 7 and 8 show a cross section through combined sample collection and filtration devices with air escape routes;

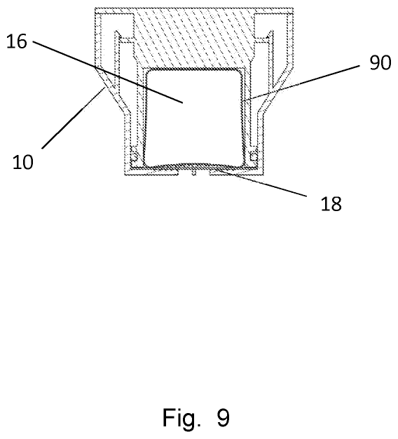

FIG. 9 shows a cross section through a combined sample collection and filtration device with a compressible bladder;

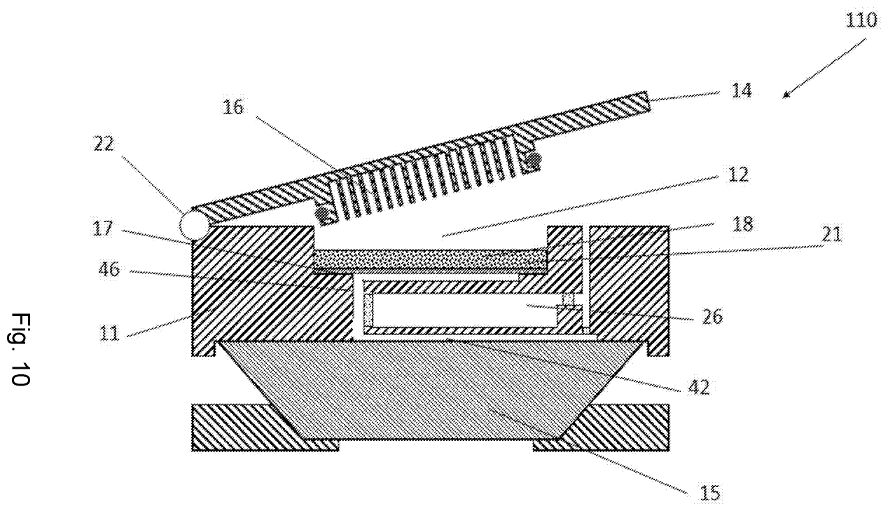

FIG. 10 shows a cross section through a combined sample collection and filtration device deployed in a hand held consumable;

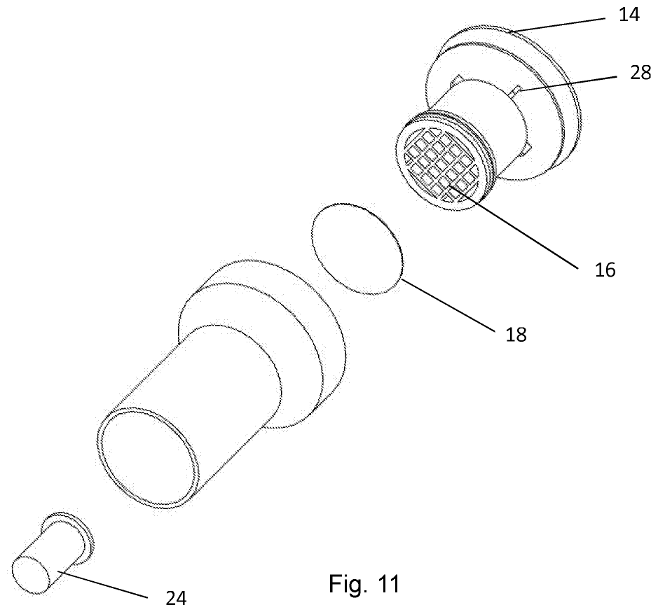

FIG. 11 shows an exploded view of a combined sample collection and filtration device deployed in a hand held consumable optimised for processing the sample prior to introducing the consumable into apparatus configured to provide high throughput analysis of multiple samples;

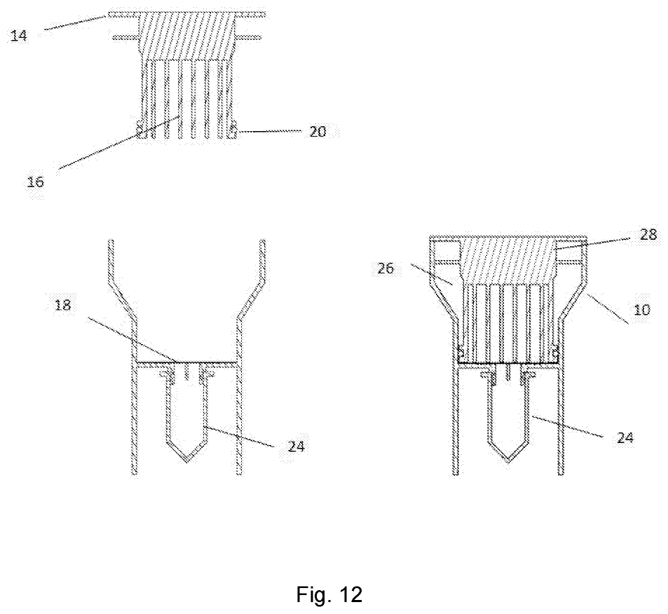

FIG. 12 shows a cross section through the high throughput consumable of FIG. 11;

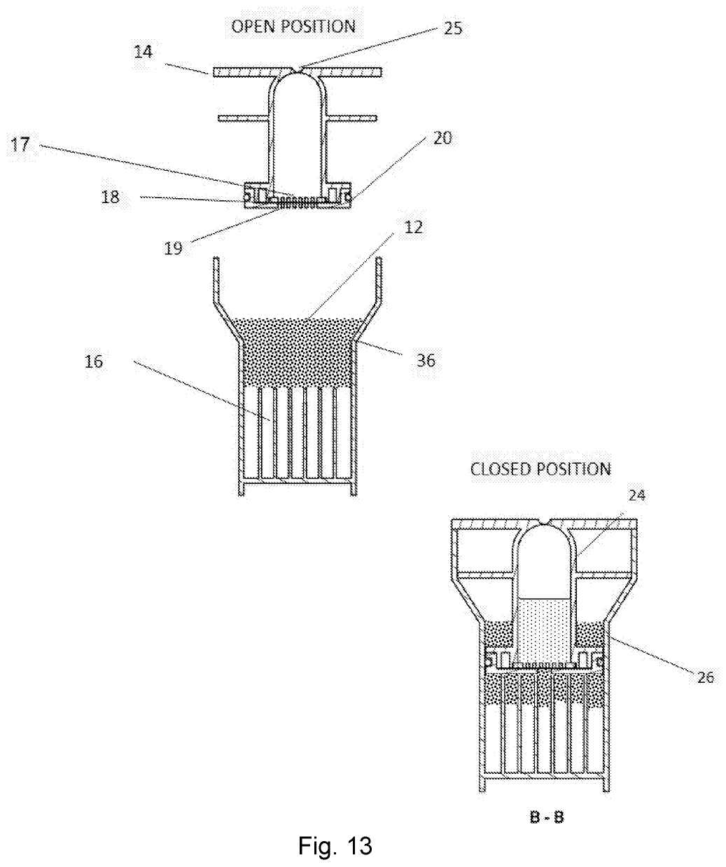

FIG. 13 shows a cross section through a combined sample collection and filtration device with voids within the main body;

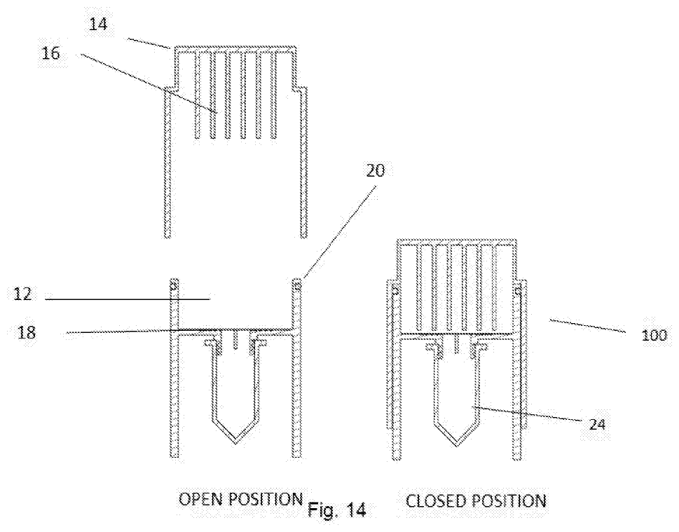

FIG. 14 shows a cross section through an alternative combined sample collection and filtration device forming part of a high throughput consumable;

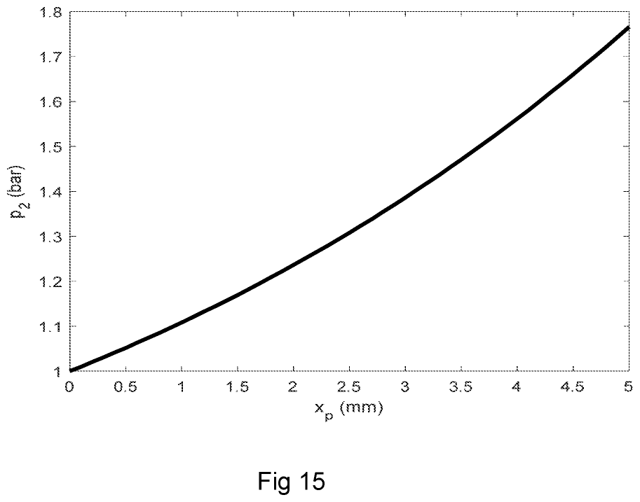

FIG. 15 shows a plot for calculating pressure of the system according to the present invention, with Xₚ (downward displacement of the plunger from the point of sealing) shown on the X-axis and P₂ (pressure in the void at the moment the lid is fully closed) shown on the Y-axis of the plot;

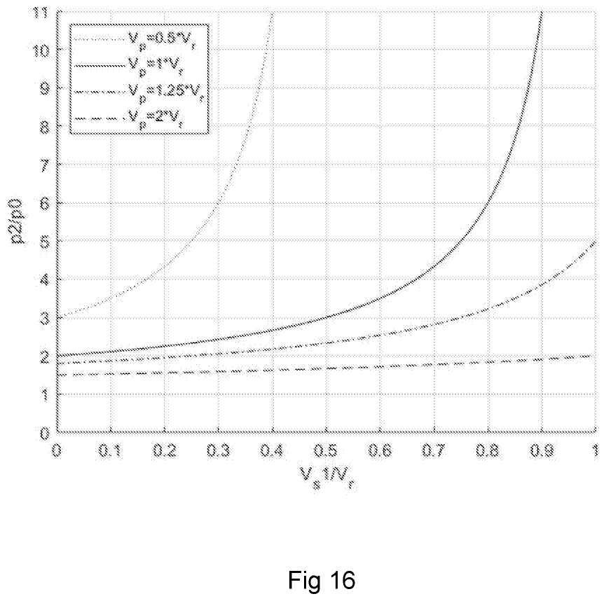

FIG. 16 shows an alternative plot for calculating pressure of the system according to the present invention, with Vₛ1/Vᵣ (volume of sample at the moment the lid seals the void/volume of the receptacle which is the entire volume of the system (V_tot) once the seal is made minus the volume in the system that can exist once the plunger is fully depressed) shown on the X-axis and p2/p0 (pressure in the void at the moment the lid is fully closed/atmospheric pressure) shown on the Y-axis of the plot; and

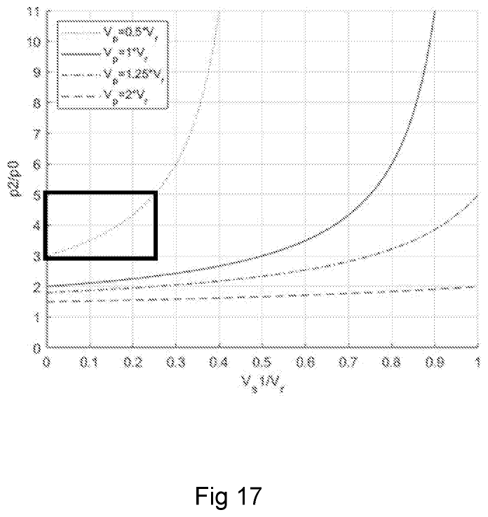

FIG. 17 shows an alternative plot for calculating pressure of the system according to the present invention, with Vₛ1/Vᵣ (volume of sample at the moment the lid seals the void/30 volume of the receptacle which is the entire volume of the system (V_tot) once the seal is made minus the volume in the system that can exist once the plunger is fully depressed) shown on the X-axis and p2/p0 (pressure in the void at the moment the lid is fully closed/atmospheric pressure) shown on the Y-axis of the plot.

Throughout the description, like reference numerals are deployed to describe similar or identical parts.

FIG. 1 shows a combined sample collection and filtration device 10 for collecting and filtering a fluid sample 27. The sample collection device 10 is bounded by a housing 11 and includes a sample collection location 12 into which the fluid sample is introduced; a lid 14 configured to cover the sample collection location 12 when the device 10 is closed; one or more voids 16 in fluid communication with the sample collection location 12 and a filter module 18.

The sample collection location 12 is an open receptacle which is generally larger in diameter than in vertical extent. The plan view of the sample collection location may be a circle, an oval or ellipse. In the later cases, where a true diameter is not defined, the major axis is larger than the vertical extent of the sample collection location 12. The shape of the collection location is chosen to be appropriate for the direct provision of a saliva sample into the device. In particular, an oval or ellipse shape when viewed from above can be advantageous as it mirrors the shape of the user's mouth.

The sample collection device 10 also includes a lid 14 configured to cover the sample collection location 12 upon closure of the device 10 after receipt of a fluid sample. The lid 14 comprises a void 16 that is in fluid communication with the sample collection location 12 upon closure of the device after receipt of a fluid sample.

The lid 14 further comprises an O-ring seal 20 to ensure a closed system after closure of the device 10. The sample collection device 10 further comprises a filter module 18 for removing particulate matter from the fluid sample. The filter module 18 has a bubble point. Assuming that the filter is pre-wetted with liquid, the bubble point refers to the maximum pressure that can be sustained within the device before gas escapes through the filter module. The bubble point is a function of pore size, filter medium wettability, surface tension and angle of contact. In particular:

$$p = \frac{4 γ cos θ}{{d}_{p}} \qquad Equation 1$$

Where
ρ=pressure difference across the filter
γ=interfacial tension of the air-sample interface
θ=wetting angle with the filter membrane
dₚ=pore diameter

Equation 1 is for a theoretical filter of symmetrical tubes and, in that case, the pressure difference across the filter is the bubble point. Equation 1 shows that the bubble point is inversely proportional to the pore diameter and therefore increasing the pore size will result in a reduction in the bubble point.

However, in practice, the filter membrane materials and any deviation from a circular pore impact the pressure difference across the filter. Sometimes, the equation is augmented by a shape correction factor. The shape correction factor is difficult to determine theoretically and, as a result, the bubble point of a filter is almost always determined empirically.

To test empirically for the bubble point; the filter membrane is first wetted with the fluid of interest and a gradually increasing gas pressure is applied to the filter membrane. At a specific gas pressure, a bubble starts forming from the largest pore first this gas pressure is defined to be the bubble point for the membrane. This empirical method is an indirect measurement of the size of the largest pore on the filter. It does not indicate the variability of pore sizes or irregularity of the membrane. In addition, the bubble point is inversely proportional to the diameter of the pore size of the membrane.

The bubble point test is somewhat sensitive to the pore length and the rate of pressure increase. The test can also give an indication of the pore size distribution. A step increase in pressure will reveal an increasing number of pores producing bubbles. Eventually a pressure is reached where the entire surface is steaming with air bubbles i.e. the “boil-point”. This is the pressure that corresponds to the mean pore size of the filter.

For example, a filter with 5 μm pores could have a bubble point at a pressure of approximately 0.5 bar whereas a filter with a 0.02 μm pore size would have a bubble point approximately 250 times higher at a pressure of 125 bar. This pressure is so high that an unskilled operative could not even reasonably likely to produce this on a small fluidic device; and even if they did, the burst pressure it would likely first rupture the fluidic membrane at the burst pressure. As a result, for designs with small pore sizes (e.g. <1 μm for the example shown) the burst pressure becomes the important pressure to limit to rather than the bubble point.

Note, to increase the burst pressure, combinations of filter membranes on supporting materials either behind (downstream of) the membrane in integrally woven into the filter membrane can increase the burst pressure of the membrane by adding additional support.

As disclosed herein, and unless otherwise specified, the term “burst pressure” refers to the pressure at which a membrane or a filter module of the device will rupture or fail. Thus, this may allow unfiltered sample to potentially break through or bypass the membrane and can have adverse effects on the results.

To increase the burst pressure, a combination of filter membranes on supporting materials can be provided downstream of the membrane such that the supporting materials can be integrally woven into the filter membrane. This can increase the burst pressure of the membrane and therefore, the membranes are more resilient and less likely to rupture.

When the device is closed, the void 16 provides a compressible sealed volume that accommodates sample during pressurisation such that the pressure within the device remains below the bubble point of the filter module 18. The sample passes through the filter and leaves the device 10 to be stored or further processed as appropriate.

The filter module 18 comprises one or more layers of filter material within the sample collection device 10. The different filters may have different pore sizes. In some embodiments, the pore size may be between 0.02 μm to 5 μm, or it may be more than 0.02, 0.05, 0.07, 0.1, 0.2, 0.4, 0.6, 0.8, 1, 2, 3 or 4 μm. It some embodiments, the pore size may be less than 5, 4, 3, 2, 1, 0.8, 0.6, 0.4, 0.2, 0.1, 0.07 or 0.05 μm.

In some embodiments, such as that illustrated in FIG. 13, the filter module 18 may include one or more support structures 17 that are configured to protect the filter, which may be brittle. Furthermore, the filter module 18 may include a protective layer that lies on top of the filter to prevent damage of the filter by the user.

The sample collection and filtration device 10 shown in FIG. 2 is similar to that shown in FIG. 1, with the exception that there are a plurality of voids 16 provided in a grid formation with seven visible in cross section. The largest dimension of each of the voids is the vertical extent so that a number of voids can be accommodated in each direction in the horizontal plane. The provision of multiple voids ensures that the sample remains distributed across the filter module 18 location during pressurisation even if the device is tipped so that the sample collection location 12 is no longer in a horizontal configuration. This provides an advantage over the example with a single void shown in FIG. 1, where the sample can migrate under gravity if the device is tipped during pressurisation. This can result in air being in contact part of the filter module 18 which, if it is not completely wetted, can provide a route for bubbles to enter the device.

FIG. 3 shows a sample collection and filtration device 10 in which the filter module 18 comprises a pad 30, a permeable protective cover 32 and a fine filter 34. The pad 30 is compressible and formed from an absorptive material. It is provided, at least in part, so that the sample does not exit the device 10 once it has entered it. The pad 30 also provides an initial filtering step with a relatively coarse grade filter, removing large particulates that could degrade the sample or damage the fine filter 34. The compressible pad will filter out large particulates that have the potential to damage or block the fine filter 34. The compressible pad 30 is capable of compressing down to around 10% of its uncompressed volume, although the compression is not required to flow the sample through the filter module 18 and out of the sample collection device 10 as the fluid will be drawn through by capillary forces on the sample. The filter module 18 may be integrally formed. In the configuration illustrated in FIG. 3, the fine filter 34 is affixed to the protection cover 32. In other examples, not illustrated, the fine filter 34 may be provided on the lower surface of the pad 30 so that the pad 30 effectively takes on the protective role of the cover 32 without providing a separate layer.

The housing 11 is provided with an indicator 36 which denotes with the maximum sample volume. The indicator 36 may be a facet of the structure or geometry of the housing 11 or it may be a visual indicator such as a line. Any sample donated over the maximum will not be pressurised as it will flow into the larger cross section part of the housing 11 above the indicator 36 and be contained within the upper part of the device 10. In this illustrated embodiment, the indicator 36 also coincides with the structural point where the seal is formed. It is the lip or shoulder where the cross section of the device changes. It will therefore be visually apparent to the user, although this point may be emphasised by a visual marking. The O-ring will not form a seal with the larger cross sectional area region above the mark 36. The pressure is therefore at least partly limited by controlling the maximum volume that can be pressurised. Any excess sample that is donated over the maximum volume will not be pressurised, but will instead be moved into one or more overflow reservoirs 26.

FIG. 4 shows a sample collection and filtration device 10 in which the actuation of the filtration step is entirely decoupled from the user's application of pressure. The lid 14 has a screw thread 15 so that the user provides rotational movement to the lid 14 to close it, rather than a downward pressure. Within the lid 14 there is a pre-compressed spring 40 and a plunger 13. The plunger 13 is retained in the lid 14 until the screw top lid is sufficiently screwed down that there is contact between the inner protrusions 42 of the lid 14 and the upper limit of the ribs 44 in the housing 11. This contact is sufficient to move the latch 46 of the lid 14 from release track 48 of the plunger 13, releasing the spring 40 which is pre-compressed. The plunger 13 then presses the fluid sample through the filter module 18. Although this configuration is more complex to manufacture, with additional parts, it does have the advantage that the pressure applied is tightly controlled as it is dictated by the spring force and totally unconnected with the user. A further advantage of providing a spring is that the maximum pressure is applied at the moment of initiation and the pressure then tails off in a predictable manner. This potentially contrasts with user operated configurations which may be deployed in a less predictable manner.

FIG. 5 shows a sample collection and filtration device 10 with a second plunger 50. The device 10 also includes a single use clip 52 that is configured to deploy when the lid 14 is introduced into the device 10. As the lid 14 is introduced into the device 10 an annular protrusion 54 on the lid, engages with the clip 52 ensuring that the lid 14 once closed cannot be opened again. This ensures that the device is a single use device. As the lid 14 is introduced into the device 10 the second plunger 50 is activated to compress the volume of air above the second plunger 50 and thereby accommodate the sample within the void 16. The sample can then be filtered by being drawn through the filter module 18 and out of the device 10 and, as it does so the second plunger 50 will return to its initial position.

FIG. 6 shows a sample collection and filtration device 10 upon completion of filtering with a deformable barrier 60. The deformable barrier 60 is configured to deform elastically as the pressure on the filter module 18 increases and can occupy a significant portion of the void. The deformable barrier 60 stores energy as it is deformed and is thus primed to release the energy and return to its un-deformed shape. This ensures that the barrier 60 stays in contact with the sample throughout, avoiding the introduction or reintroduction of any air at the filter module 18 interface. The elasticity of the deformable barrier will be selected to ensure that the pressure within the device 10 never exceeds the bubble point of the filter module 18.

FIG. 7 shows a sample collection and filtration device 10 upon completion of filtering with a spring moderated second plunger 50 and an air escape route 70. The air escape route 70 is a narrow tube that protrudes through the lid 14 to enable air from the void 16 to move out of the device 10. Once the air has escaped, the return of the second plunger 50 to its undeformed position relies on the spring force of the spring which has been compressed as the second plunger 50 has depressed into the void 16 to prevent the pressure of the sample from overwhelming the filter module 18 and allowing bubbles to form.

FIG. 8 shows a sample collection and filtration device 10 that has an air escape route 70 as described above with reference to FIG. 7 and a deformable barrier 60 as described above with reference to FIG. 6.

FIG. 9 shows a sample collection and filtration device that has a compressible bladder 90 provided in the void 16. The compressible bladder provides a continuous interface with the sample surface regardless of the angle of the device. The bladder is then capable of compressing during pressurisation of the device 10.

FIG. 10 shows a sample collection and filtration device 10 incorporated into diagnostics device 110 designed for use by the non-skilled user in the home, workplace, domestic or retail setting. The housing 11 is configured to encapsulate the sample collection device 10 including the filter module 18. One or more detection reagents 21 are provided in the filter module 18. As the salvia sample passes through the filter module 18 large particulates are caught by the filter and removed from the saliva sample. At the same time, the detection reagents 21 are introduced into the saliva sample so that they pass together throughout the filter module 18. The housing 11 provides an interface for the lid 14 enabling the creation of a closed unit when the lid 14 is brought into contact with the housing 11. The shape and configuration of the outer surface of the housing 11 can be selected to closely conform to the sample collection device 10 and an optical element, such as a prism 15 which is provided below the filtration module 18. On the upper surface of the prism 15 are deposited one or more capture components 42. When the saliva sample has left the sample collection and filtration device 10 via the filter module 18, it flow along a fluid pathway 46 and then across the upper surface of the prism 15 and contacts the capture components 42. The detection reagents 21 and capture components 42 form a sandwich assay around the biomarker of interest within the saliva sample. The fluid pathway 46 is tortuous so that the filter module 18 is effectively masked from the capture components 42.

The lid 14 is attached to the device by a hinge 22. This ensures that the lid 14 can pivot clear of the sample collection location to give the user the maximum ease of use in relation to providing the sample. In addition, the provision of a hinged lid ensures that the lid cannot be dropped or lost by the user. This is particularly critical when the device is designed for use in a non-sterile environment such as a home, workplace, domestic or retail setting by an unskilled user.

FIGS. 11 and 12 show a combined sample collection and filtration device 10 for collecting and filtering a fluid sample that is provided as part of a cartridge 100. The cartridge 100 is optimised for collection and filtration of a saliva sample and then subsequently storing the filtered sample ready for further processing. This type of cartridge is designed for collection of the sample at the point of care and then subsequent processing of the sample in a high throughput device, potentially at a remote location. It is therefore important that the sample is effectively filtered at the time of collection to remove particulate matter that could degrade the sample over time and prevent effective processing of the sample when assays are subsequently run in a high throughput device.

The cartridge 100 is provided with a sample collection and filtration device 10, an overflow reservoir 26 and a sample storage tube 24. The housing 11 encapsulates all of the features of the cartridge 100 and has a circular cross section and a lid 14 that is not hinged.

The overflow reservoir 26 is provided for accommodating excess fluid sample. The lid 14 is provided with a plurality of voids 16 that accommodate the sample during the closure of the cartridge 100. If the volume of sample exceeds the volume that can be accommodated within the voids 16, then the excess volume moves into the overflow reservoir 26. This is important because any volume of sample that exceeds the maximum volume of fluid that is required must not exit the cartridge where is risks cross contaminating other samples by contacting the reader into which the cartridge 100 is inserted for analysis and read out of assay results. In the configuration illustrated in FIGS. 11 and 12, the overflow reservoir 26 is defined by a larger cross section volume above the sample collection location 12. In the corresponding larger cross sectional area region of the lid 14 there are four tines 28 each occupying a radial quarter of the lid 14. Although this example is illustrated with four radial tines, similar configurations with three, five, six or up to 12 radial tines 28 can be envisaged. The tines 28 provide structural integrity to the lid 14 to ensure that it does not deform during its deployment to close the cartridge 100.

The device 10 further comprises a storage tube 24 downstream of the filter module for storing processed fluid sample. Once the sample reaches the storage tube 24 it remains in the storage tube 24 until the sample reaches its intended destination for further processing and analysis. The storage tube 24 has a cylindrical form, but it could take any appropriate shape. The storage tube 24 is not in direct contact with the housing 11 of the cartridge 100 so that the sample is insulated from the environment around the cartridge 100 whilst the cartridge is in transit. This includes thermal insulation from the environment and also potential for damage from impacts to the housing 11 of the cartridge 100.

FIG. 13 shows an alternative embodiment of a cartridge 100 for collection and filtration of a sample in preparation for running the assays on the samples in a high throughput device at a remote location at a subsequent time. In this embodiment the configuration of the parts is inverted with the storage tube 24 being provided in the lid 14. The upper surface of the lid 14 is provided with a frangible point 25 which can be pierced in order to remove the filtered sample from the storage tube 24. The filter module 18 includes a guard 19 and a support structure 17.

The guard 19 provides protection for the filter from the user. The guard 19 is positioned upstream of the filter module 18, for example, in the sample collection location 12 so that the user does not have direct access to the filter module 18.

The filter membrane(s) of the filter module 18 has a positive wetting coefficient and wicks in the liquid sample forming a seal. The positive wetting coefficient of the filter membrane ensures that, as the liquid sample comes into contact with the filter membrane, it wets the surface and through the filter membrane ensuring that no bubbles become entrained in the sample at the interface with the filter module 18. The positive wetting coefficient may be inherent to the filter material or it may be provided by a coating or treatment.

FIG. 14 shows an alternative combined sample collection and filtration device 10 for collecting and filtering a fluid sample that is provided as part of a cartridge 100. The cartridge 100 is optimised for collection and filtration of a saliva sample and then subsequently storing the filtered sample ready for further processing in line with the embodiments described above with reference to FIGS. 11 and 12.

The cartridge 100 shown in FIG. 14 has no overflow. Instead, the user fills the sample collection location 12 to either the top or to the indicator 36 which may be a visible line below the upper surface of the housing 11. The O-ring 20 is provided on the outer surface of the housing 11 to provide a seal between the housing 11 and lid 14 as soon as the lower edge of the lid 14 passes over the O-ring 20 during closure. In other configurations, not illustrated in the accompanying drawings, the seal may not be provided by an O-ring, but, instead may be achieved through an interference fit between the lid and the housing.

The benefit of this design is that the void volume can be compressed more than the volume of the maximum sample during closure. This means there is more pressure to filter the last portion of sample as there will residual pressure in the device even after the sample has been filtered.

### EXAMPLES

### Example 1—Lid Closure: Equation for the Pressure in the Void

A series of equations as shown below are provided to demonstrate how the pressure in the system as disclosed in the present invention is calculated to allow parameters including the volume of voids and spring constants or compliance of materials to be tuned. Calculating the pressure within the system enables a limit to be set on the pressure within the system as the main plunger first makes a seal, then travels to a final point at which there is a residual volume of the system V_p.

The following calculations are made using various approximations and assumptions as set out below. Despite these approximations and assumptions, the equations that follow are sufficiently applicable to be used for all illustrated embodiments. The equations do not take into account how the volume of sample V_s can vary as the plunger is depressed, as during depression some of the sample can actually pass through the filter membrane at a rate proportional to the pressure differential across the filter membrane (and the pore size and material of the membrane and viscosity of the liquid). The filter can also clog with particulate matter as the sample is passed through it, adding to the resistance of the filter and slowing the rate of filtration. In the following simplified equation, the volume of sample when fully depressed is defined as a parameter V_s2. In addition, the boundary or ‘worst case’ condition (from a high pressure point of view) where there is no filtration of sample over the time take for the plunger to fully be depressed can be investigated. This filtration rate could be explored and accounted for in order to limit the pressure in the system in a less conservative manner than the worst case exemplified.

It should be noted that although the equations incorporate a second plunger with a spring, as illustrated in FIG. 7, the spring constant ‘k’ can be replaced by one or more of the following: a deformable membrane as shown in FIG. 8; a sealed compressible void with second plunger as shown in FIG. 5, where the spring constant comes from the ideal gas law as the volume is compressed; a membrane that can deform with elastic spring constant k, which may also vary as a function of the deformation; and a design with both a deformable membrane and a sealed void as shown in FIG. 6.

### Assumptions

One or more assumptions are made during the calculation of pressure in the system as described below:
during the air compression, the air behaves as an ideal gas, and the compression is isothermal.
inertia of the sample is ignored
the compliance in the plunger is modelled as a piston on a spring (inside the larger piston that is the plunger). As stated above this could be modelled as per other embodiments.
friction effects that might stop the second plunger from complying which could be modelled as additional resistance.

As disclosed herein, and unless otherwise specified, the worst case scenario in this context refers to the fact that the sample without any bubbles can be filled to the top and an instantaneous lid closure can occur, such that the compressible volume is filled rather than any liquid going through the filter membrane before the lid is fully closed.

### Derivation of Equation

Total Void Volume:

Vᵥ=Vᵣ−Aᵣxₚ+Vₚ+V_{c}

V_{g}=Vᵥ−Vₛ

V_{g1}=Vᵣ+Vₚ−Vₛ₁

V_{g2}=Vᵣ−AᵣXₚ+Vₚ+V_{c}(p₂)−Vₛ₂

From ideal gas law and isothermal air compression:

p₂V_{g2}=p₁V_{g1}

p₂[Vᵣ−Aᵣxₚ+Vₚ+V_{c}(p₂)−Vₛ₂]=p₁[Vᵣ+Vₚ−Vₛ₁]

p₁=p₀

Compliance displacement x_{c} (when modelled as a plunger):

$${x}_{c} = \frac{( {p}_{2} - {p}_{o} ) {A}_{c}}{k}$$

$${V}_{c} ( {p}_{2} ) = {A}_{c} {x}_{c} ( {p}_{2} ) = \frac{( {p}_{2} - {p}_{o} ) {A}_{c}^{2}}{k}$$

$${p}_{2} [ {V}_{r} - {A}_{r} {x}_{p} + {V}_{p} + \frac{( {p}_{2} - {p}_{o} ) {A}_{c}^{2}}{k} - {V}_{s 2} ] = {p}_{o} [ {V}_{r} + {V}_{p} - {V}_{s 1} ]$$

$${p}_{2} \frac{( {p}_{2} - {p}_{o} ) {A}_{c}^{2}}{k} + {p}_{2} ( {V}_{r} - {A}_{r} {x}_{p} + {V}_{p} - {V}_{s 2} ) - {p}_{o} [ {V}_{r} + {V}_{p} - {V}_{s 1} ] = 0$$

$${p}_{2}^{2} \frac{{A}_{c}^{2}}{k} + {p}_{2} ( {V}_{r} - {A}_{r} {x}_{p} + {V}_{p} - {V}_{s 2} - {p}_{o} \frac{{A}_{c}^{2}}{k} ) - {p}_{o} [ {V}_{r} + {V}_{p} - {V}_{s 1} ] = 0$$

$${ap}_{2}^{2} + {bp}_{2} + c = 0$$

$$a = \frac{{A}_{c}^{2}}{k}$$

$$b = ( {V}_{r} - {A}_{r} {x}_{p} + {V}_{p} - {V}_{s 2} - {p}_{o} \frac{{A}_{c}^{2}}{k} )$$

$$c = - {p}_{o} [ {V}_{r} + {V}_{p} - {V}_{s 1} ]$$

### General Solution:

Where

$${p}_{2} = \frac{- b \pm √ ( {b}^{2} - 4 a c )}{2 a} \qquad Equation ( 2 )$$

p0: atmospheric pressure;
p1: pressure in the void at moment of sealing the lid (during closing);
p2: pressure in the void at the moment the lid is fully closed;
V_c: compliant wall's void expansion;
A_r: the horizontal cross-sectional area of the receptacle;
h_r: height of receptacle assuming uniform cross section;
V_r: volume of the receptacle which is the entire volume of the system (V_tot) once the seal is made minus the volume in the system that can exist once the plunger is fully depressed;
x_p: the downward displacement of the plunger from the point of sealing;
V_p: internal volume of the system defined by the geometry when the plunger in in the fully depressed (closed) position. By way of example only, in FIG. 7, this is zero, In another example as illustrated in FIGS. 1,2,3,12,13 &14 this includes the additional compressible volume but not any compliance of plungers or membranes;
V_s1: volume of sample at the moment the lid seals the void;
V_s2: volume of sample at the moment the lid fully closes;
A_c: Cross sectional area of the compliance in the second plunger;
k: spring constant of the compliance.

Referring to FIG. 15, there is provided a plot based on Equation 2 and varying x_p from 0 to 5 [mm] whilst keeping all other parameters fixed at; k=1000 [N/m], V_r=5e-6[m\hat{ }3]; A_r=5e-4 [m\hat{ }2]; V_s1=1e-6 [m\hat{ }3]; V_s2=1e-6 [m\hat{ }3]; p0=1e5 [Pa]; V_p=0. The plot shows the positive solution only from the quadratic. Example of pressure in the system based on assumptions as disclosed herein over a travel of 5 mm from the point at which a seal is made to the point of closure.

### Example 2—Simplification of Embodiments Illustrated in FIGS. 1, 2, 3, 12, 13 and 14

The following is for cases with no second plunger or deformable membrane (i.e. compressible void(s)). This is a preferred embodiment of the invention as disclosed herein because it is simple and therefore cheap to manufacture. It is also easier to operate because there are no extra parts or moveable parts.

The simplified case with compressible void, but without barrier or second plunger are illustrated in embodiments as shown in FIGS. 1, 2, 3, 12, 13 and 14. In this case there is effectively no displacement xc thus k approaches infinity; although slight deformation of components such as injection moulded parts could be considered compliance and modelled.

; xc=0 thus k=infinity

$${p}_{2} = {p}_{o} \frac{[ {V}_{r} + {V}_{p} - {V}_{s 1} ]}{[ {V}_{r} - {A}_{r} {x}_{p} + {V}_{p} - {V}_{s 2} ]}$$

If the design could allow a fully compressed plunger when fully compressed such that Aᵣxₚ=Vr

$${p}_{2} = {p}_{o} \frac{[ {V}_{r} + {V}_{p} - {V}_{s 1} ]}{[ {V}_{p} - {V}_{s 2} ]}$$

It can be seen at a worst case where, assuming that no sample gets filtered through the membrane Vₛ₁=Vₛ₂

$$\frac{{p}_{2}}{{p}_{o}} = \frac{[ {V}_{r} + {V}_{p} - {V}_{s 1} ]}{[ {V}_{p} - {V}_{s 1} ]} \qquad Equation ( 3 )$$

Also; assuming that the sample fills the receptacle fully with no air or bubbles as is possible in designs apart form that shown in FIG. 14

$$Vr = {V}_{s 1}$$

$$\frac{{p}_{2}}{{p}_{o}} = \frac{[ {V}_{p} ]}{[ {V}_{p} - {V}_{s 1} ]} \frac{{p}_{2}}{{p}_{o}} = \frac{1}{[ 1 - \frac{{V}_{s 1}}{{V}_{p}} ]} \qquad Equation ( 4 )$$

Referring to FIG. 16, there is shown a plot based on Equation 3, with a fixed V_s1 of 5e-6 and varying V_s1 from 0 to V_r, equation 3 is shown for illustration at different V_p values ranging from 0.5*V_r to 2*V_r and plotted. As ratios are shown, the exact value of V_r and thus all other parameters are not important as long as it is >0.

As illustrated in FIG. 16, the plot shows the ratio of pressure increase above the initial (ambient) pressure depending on the ratio vs the ratio of the sample to the receptacle volume (V_s1/Vr), with assumption that no saliva is filtered through the membrane and that the plunger is fully depressed.

FIG. 16 shows p2/p0 axis is cut off at 11, but the pressure ratio increases to infinity as the sample is considered an incompressible liquid for ratios of V_p/V_r<1. This shows that to ensure pressure is maintained below the bubble point of the filter for embodiments without a second plunger or deformable membrane, i.e. embodiments as illustrated in FIGS. 1, 2, 3, 12, 13 and 14, the volume in the system at full compression must be greater than the volume of sample.

Referring to FIG. 17, there is shown a plot for calculating the pressure within the system according to the present invention, where the sample is introduced in the sample collection unit. In contrast, the embodiment shown in FIG. 14 is by a different by design because at the point when a seal is made the ratio of possible sample volume to the receptacle volume can be chosen as a ratio much lower than 1, for example about 1/4. This is because a lot of the receptacle volume V_r is defined by the lid or plunger component rather than the sample collection unit. Referring to FIG. 17, as long as sample is only introduced into the sample collection unit and not the lid or plunger then this ratio can be tuned and the pressure in the system can be increased whilst still being kept below the bubble point as shown by the solid box area in FIG. 17.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

1. A combined sample collection and filtration device for collecting and filtering a fluid sample, the device comprising:
a sample collection location;
a filter module for removing particulate matter from the fluid sample, the filter module having a bubble point and a burst pressure; and
one or more voids in communication with the sample collection location for accommodating a fluid sample during pressurisation such that the pressure within the device remains below the bubble point and the burst pressure of the filter module.

2. (canceled)

3. The sample collection device according to claim 1, wherein there is a plurality of voids separated by a plurality of partitions.

4. The sample collection device according to claim 1, wherein the or each void comprises a sealed volume that is compressed upon closure of the device.

5. The sample collection device according to claim 1, wherein the device comprises a compressible absorptive pad.

6. The sample collection device according to claim 5, wherein the compressible absorptive pad is removable.

7. The sample collection device according to claim 1, further comprising a closure with a one-way latch.

8. The sample collection device according to claim 1, wherein the filter module comprises a plurality of filter-membranes and wherein porosity is graded to prevent membrane clogging.

9. The sample collection device according to claim 8, further comprising a support structure residing between the filter membrane(s) and the compression compartment.

10. The sample collection device according to claim 8, further comprising a guard.

11. The sample collection device according to claim 8, wherein the filter membrane(s) of the filter module has a positive wetting coefficient and is configured to wick in the fluid sample and to thereby form a seal.

12. The sample collection device according to claim 1, wherein the device further comprises a check-valve in fluid communication with the filter module.

13. The sample collection device according to claim 7, further comprising a stabilising agent.

14. The sample collection device according to claim 7, further comprising a detection reagent.

15. The sample collection device according to claim 14, wherein the stabilising agent and detection reagent are co-located.

16. The sample collection device according to claim 15, wherein the detection reagent is a peptide, a protein, a protein assembly, an oligonucleotide, a polynucleotide, a modified oligonucleotide, a modified polynucleotide, an aptamer, a morpholino, a small molecule, a cell, a cell membrane, a viral particle, a glycan, a conjugated solid particle, a conjugated solid bead, or a cofactor, and wherein the detection reagent is labelled with a luminophore, a fluorophore, a phosphor, a chemiluminescent molecule, an enzyme, or an up-conversion particle.

17. The sample collection device according to claim 1, wherein the one or more voids further comprises a plunger element for separating fluid sample from gas in the one or more voids and for preventing the fluid sample from flowing off the filter module.

18. The sample collection device according claim 17, wherein the one or more voids comprise an air vent configured to allow air to escape the device.

19. The sample collection device according to claim 1, wherein the device further comprises a deformable barrier to the one or more voids for separating fluid sample from gas in the one or more voids and for preventing the fluid sample from flowing off the filter module.

20. The sample collection device according to claim 19, wherein the one of the voids comprises an air vent configured to allow air to escape the device.

21. The sample collection device according to claim 1, wherein one of the voids comprises a bladder of compressible gas for separating fluid sample from gas in the one or more voids and for preventing the fluid sample from flowing off the filter module.
